(19) United States
(12) Patent Application Publication
(10) Pub. No.: US 2025/0121175 A1
(43) Pub. Date: Apr. 17, 2025

(54) DISTAL EXTENSION FOR BLOOD PUMP SYSTEMS

(71) Applicant: ABIOMED, Inc., Danvers, MA (US)

(72) Inventors: Christopher Zarins, Danvers, MA (US); Ralph D'Ambrosio, Danvers, NJ (US); Max Solar, Danvers, NJ (US)

(73) Assignee: ABIOMED, Inc., Danvers, MA (US)

(21) Appl. No.: 18/912,769

(22) Filed: Oct. 11, 2024

Related U.S. Application Data
(60) Provisional application No. 63/543,962, filed on Oct. 13, 2023.
(60) Provisional application No. 63/696,192, filed on Sep. 18, 2024.

(51) Int. Cl. A61M 60/13; A61M 60/216; A61M 60/408

(52) U.S. Cl. CPC A61M 60/13; A61M 60/216; A61M 60/408

(57) ABSTRACT

A distal extension for a blood pump may be provided. The distal extension may include a tubular body. The distal end of the tubular body may be coupled to an intermediate point on the tubular body to form a closed loop at a distal end of an atraumatic distal extension. The closed loop may define an aperture extending through the closed loop, the closed loop having a central plane parallel to a central axis of the tubular body at the proximal end. In various aspects, the tubular body may include a bend. The bend may define an angle between a central axis of a first substantially straight section of the tubular body proximal to the bend and a central axis of a second substantially straight section of the tubular body distal to the bend. In various aspects, the closed loop may be a non-circular closed loop.

### CROSS-REFERENCE TO RELATED APPLICATIONS

The present application claims priority to U.S. Provisional Applications Nos. 63/543,962, filed Oct. 13, 2023, and 63/696,192, filed Sep. 18, 2024, each of which is incorporated by reference herein by its entirety.

### TECHNICAL FIELD

The present disclosure is drawn to distal extensions for blood pump systems.

### BACKGROUND

A blood pump assembly (e.g., an intracardiac or intravascular blood pump) may be introduced into the heart to deliver blood from the heart into the artery. Such mechanical circulatory support devices are typically introduced to support the function of the heart after a patient has suffered a heart attack. One class of devices is a suite of devices known as “ventricular assist devices” (VADs) or blood pumps. Some blood pump assemblies may be introduced percutaneously through the vascular system during cardiac surgery. Specifically, the blood pump assembly may be, e.g., inserted into the ascending aorta via a femoral artery or an axillary/subclavian artery via catheterization, through the valve and into the left ventricle. However, once the blood pump is in a patient, small structures (including, e.g., valves) within the patient can get stuck on or damaged by conventional distal extensions.

### BRIEF SUMMARY

In various aspects, a distal extension for a blood pump may be provided. The distal extension may include a tubular body extending from a proximal end to a distal end. The distal end may be coupled to an intermediate point on the tubular body, between the proximal end and the distal end, to form a closed loop at a distal end of an atraumatic distal extension. The closed loop may define an aperture extending through the closed loop. The closed loop may have a central plane parallel to a central axis of the tubular body at the proximal end.

In certain aspects, the tubular body may include a bend. The bend may define an angle between a central axis of a first substantially straight section of the tubular body proximal to the bend and a central axis of a second substantially straight section of the tubular body distal to the bend. In certain aspects, the closed loop may be a non-circular closed loop.

A central axis of the aperture may extend through the closed loop in a direction perpendicular to the central plane of the closed loop. The aperture may be circular. The aperture may be non-circular. The central axis of the aperture may be off-center within the closed loop. The central axis of the aperture may be centered within the closed loop.

A diameter or thickness of the tubular body at the proximal end may be different from a diameter or thickness of the tubular body in the closed loop. A diameter or thickness of the tubular body at the proximal end may be equal to a diameter or thickness of the tubular body in the closed loop. A diameter or thickness of the tubular body at a first point of the closed loop may be different from a diameter or thickness of the tubular body at a second point of the closed loop. The tubular body may have a constant thickness or diameter along the entire closed loop.

A distal end surface of the tubular body may be coupled to an outer surface of the tubular body at the intermediate point. The intermediate point may be located proximal to the bend. The intermediate point may be located distal to the bend.

The distal extension may have a proximal portion with a stiffness greater than that of a distal portion. The angle defined by the bend may be at least 135 degrees and less than 180 degrees. The closed loop may be defined by a continuous curve. The closed loop may be circular. The closed loop may include one or more substantially straight sections. The closed loop may include a first point, furthest from the intermediate point, that has a radius of curvature that is less than a radius of curvature at a second point closer to the intermediate point than the first point. A first point, furthest from the proximal end may have a radius of curvature that is less than a radius of curvature at a second point closer to the proximal end than the first point.

The distal extension may include a lumen extending through at least a portion of the atraumatic distal extension. The lumen may be configured to receive a guidewire. The distal extension may be free of a lumen configured to receive a guidewire.

In some embodiments, the closed loop may be circular. In other embodiments, the closed loop may be non-circular.

In various aspects, a system may be provided. The system may include a pump section having a proximal end and a distal end. The pump section may be configured to cause blood to flow from a blood inlet, through a cannula, to a blood outlet. The system may include an embodiment of a distal extension as disclosed herein operably coupled to a distal end of the pump section.

The system may include a motor section. The motor section may be operably coupled to the pump section via a drive shaft. The system may include a catheter operably coupled to a proximal end of the pump section. In certain embodiments, the motor section may be coupled to a proximal end of the pump section and the catheter may be coupled to a proximal end of the motor section. In certain embodiments, the catheter may be disposed between the pump section and the motor section.

In various aspects, a distal extension for a blood pump may be provided. The distal extension for the blood pump may include a tubular body extending from a proximal end to a distal end. The distal end may be coupled to an intermediate point on the tubular body between the proximal end and the distal end to form a non-circular closed loop at a distal end of an atraumatic distal extension. The non-circular closed loop may define an aperture extending through the non-circular closed loop. The non-circular closed loop may have a central plane parallel to a central axis of the tubular body at the proximal end.

In some embodiments, a central axis of the aperture extends through the non-circular closed loop in a direction perpendicular to the central plane of the non-circular closed loop. The aperture may be circular. The aperture may be non-circular. In certain aspects, a central axis of the aperture may be off-center within the non-circular closed loop. A central axis of the aperture may be centered within the non-circular closed loop.

In certain embodiments, a diameter or thickness of the tubular body at the proximal end may be different from a diameter or thickness of the tubular body in the non-circular closed loop. A diameter or thickness of the tubular body at a first point of the non-circular closed loop may be equal to a diameter or thickness of the tubular body at a second point of the non-circular closed loop. A diameter or thickness of the tubular body at a first point of the non-circular closed loop may be different from a diameter or thickness of the tubular body at a second point of the non-circular closed loop. The tubular body may have a constant thickness or diameter along all of the non-circular closed loop.

In certain embodiments, a distal end surface of the tubular body may be coupled to an outer surface of the tubular body at the intermediate point. The intermediate point may be located proximal to the bend. The intermediate point may be located distal to the bend.

In certain embodiments, the distal extension may have a proximal portion that may have a stiffness greater than that of a distal portion. In certain embodiments, the angle defined by the bend may be at least 135 degrees and less than 180 degrees.

In certain embodiments, the closed loop may be defined by a continuous curve. The closed loop may include one or more substantially straight sections. The closed loop may include a first point, furthest from the intermediate point, that may have a radius of curvature that is less than a radius of curvature at a second point closer to the intermediate point than the first point. A first point, furthest from the proximal end, may have a radius of curvature that is less than a radius of curvature at a second point closer to the proximal end than the first point.

In certain embodiments, the distal extension may include a lumen extending through at least a portion of the atraumatic distal extension. The lumen may be configured to receive a guidewire. The distal extension may be free of a lumen configured to receive a guidewire.

In various aspects, a system may be provided. The system may include a pump section. The pump section may include a proximal end and a distal end. The pump section may be configured to cause blood to flow from a blood inlet through a cannula to a blood outlet. The system may include an embodiment of a distal extension as disclosed herein operably coupled to the distal end of the pump section.

In certain embodiments, the system may include a motor section. The motor section may be operably coupled to the pump section via a drive shaft. In some embodiments, the system may further include a catheter. The catheter may be operably coupled to a proximal end of the pump section. The motor section may be coupled to a proximal end of the pump section. The catheter may be coupled to a proximal end of the motor section. The catheter may be disposed between the pump section and the motor section.

In various aspects, a method of inserting a blood pump may be provided. The method may include introducing a system, which may include a blood pump, to a blood vessel, the blood pump having an embodiment of a distal extension as disclosed herein disposed at a distal end of the blood pump. The method may include advancing the system through the blood vessel, without use of a guidewire, and allowing the distal extension to interact with at least one valve, until the blood pump is disposed in a desired location.

In various aspects, a distal extension for a blood pump may be provided. The distal extension for the blood pump may include a tubular body. The tubular body may extend from a proximal end to a distal end. The tubular body may define a helical structure around an axis, where, from a proximal end point to a distal end point of the tubular body, the helical structure completes n turns, where n is at least 0.25.

In certain embodiments, the tubular body may curl clockwise, from the proximal end to the distal end, around the axis to form the helical structure. The tubular body may curl counterclockwise, from the proximal end to the distal end, around the axis to form the helical structure.

In certain embodiments, protrusions may extend from the surface of the tubular body to form the helical structure. In certain embodiments, n is at least 1. In some embodiments, n may be no more than 3. In some embodiments, n is no more than 1.

In certain embodiments, the pitch of the helical structure may be at least three times a diameter or thickness of the tubular body. In certain embodiments, the pitch of the helical structure may be no more than 10 times a diameter or thickness of the tubular body. The distal extension may be free of a lumen configured to receive a guidewire.

In various aspects, a system may be provided. The system may include a pump section. The pump section may include a proximal end and a distal end. The pump section may be configured to cause blood to flow from a blood inlet through a cannula to a blood outlet. The system may include an embodiment of a distal extension as disclosed herein operably coupled to the distal end of the pump section.

In certain embodiments, the system may further include a motor section. The motor section may be operably coupled to the pump section via a drive shaft. The system may further include a catheter operably coupled to a proximal end of the pump section. The motor section may be coupled to a proximal end of the pump section. The catheter may be coupled to a proximal end of the motor section. The catheter may be disposed between the pump section and the motor section.

In various aspects, a method of inserting a blood pump may be provided. The method may include introducing a guide catheter to a blood vessel. The method may include introducing a blood pump to a blood vessel, the blood pump having a distal extension with a helical structure as disclosed herein disposed at a distal end. The method may include advancing the blood pump through the blood vessel, without use of a guidewire. The method may include rotating the blood pump to cause the helical structure to engage the guide catheter. The method may include rotating the blood pump to cause the helical structures to interact with one or more leaflets of the aortic valve. The method may include removing the guide catheter.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1A is an illustration of a blood pump with a distal extension.

FIG. 1B is an illustration of a top view of a distal extension.

FIGS. 2A-2F are illustrations of side views of various embodiments of distal extensions.

FIG. 3 is an illustration of a blood pump positioned within a heart.

FIG. 4A is an illustration of a helical distal extension.

FIG. 4B is an illustration of a front view of a helical distal extension.

FIGS. 5-6 are illustrations of various embodiments of a helical distal extension.

FIG. 7 is an illustration of a helical distal extension engaging a guide catheter.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

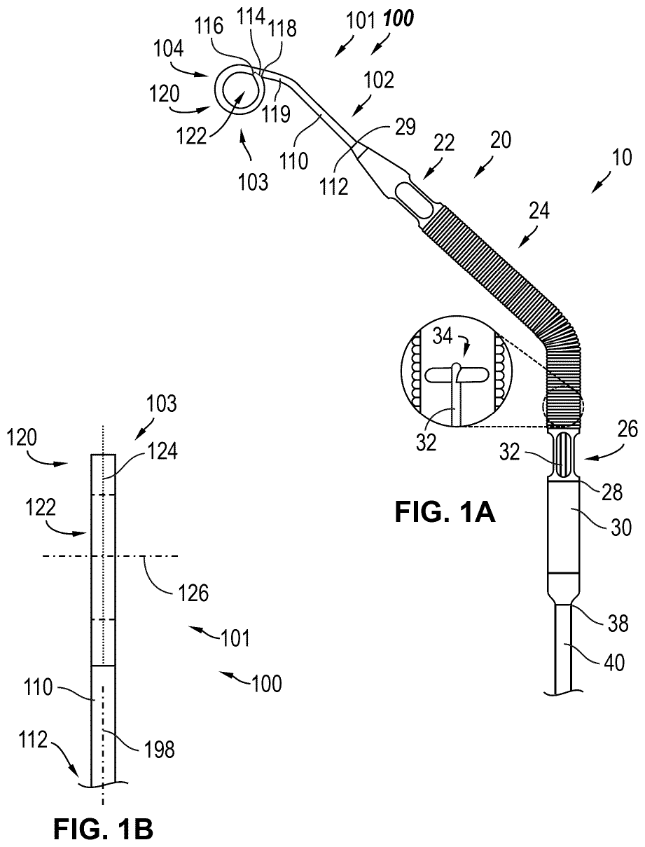

Referring to FIG. 1A, systems that incorporate blood pumps (10) may be provided. Blood pumps may include a pump section (20). The pump section may have a proximal end (28) and a distal end (29). The pump section may be configured to cause blood to flow from a blood inlet (22) through a cannula (24) to a blood outlet (26).

In some embodiments, the blood pump (10) shown in FIG. 1A may be adapted for left heart support, in accordance with aspects of the disclosure. In such embodiments, the blood pump (10) may be inserted percutaneously. For example, when used for left heart support, the blood pump (10) may be inserted via a catheterization procedure through the femoral artery or axillary artery, into the aorta, across the aortic valve, and into the left ventricle. Once positioned in this way, the blood pump (10) may deliver blood from the blood inlet (22), which sits inside the left ventricle, through cannula (24), to the blood outlet (26), which sits inside the ascending aorta.

In other embodiments, the blood pump (10) shown in FIG. 1A may be adapted for right heart support, in accordance with aspects of the disclosure. In such embodiments, the blood pump (10) may also be inserted percutaneously. For example, when used for right heart support, the blood pump (10) may be inserted via a catheterization procedure through the femoral vein, into the inferior vena cava, through the right atrium, across the tricuspid valve, into the right ventricle, through the pulmonary valve, and into the pulmonary artery. Once positioned in this way, the blood pump (10) may deliver blood from the blood inlet (22), which sits inside the inferior vena cava, through cannula (24), to the blood outlet (26), which sits inside the pulmonary artery. In some embodiments, the blood pump (10) may be inserted via the internal jugular (IJ) vein. For example, in such embodiments, the blood pump (10) may be inserted via a catheterization procedure through the IJ vein, into the superior vena cava, through the right atrium, across the pulmonary valve, and into the pulmonary artery. Once positioned in this way, the blood pump (10) may deliver blood from the blood inlet (24), which sits inside the superior vena cava, through the cannula (24), to the blood outlet (26), which sits inside the pulmonary artery.

The blood pump may include a motor section (30). The motor section may be operably coupled to the pump section via a drive shaft (32). The drive shaft may be operably coupled to an impeller (34). The impeller may be disposed within the pump section and may be configured to cause blood to flow through the pump section when the impeller is rotated. In some embodiments, at least a portion of the drive shaft (32) may be flexible.

The blood pump may include a catheter (40) operably coupled to a proximal end of the pump section. In some embodiments, such as that shown in FIG. 1A, the catheter is indirectly coupled to the pump section. More specifically, in some embodiments, the motor section may be coupled to a proximal end of the pump section, and the catheter may be coupled to a proximal end (38) of the motor section. In some embodiments (not shown), the catheter may be disposed between the pump section and the motor section. In such embodiments (not shown), the motor section may be disposed outside of the patient. In some embodiments (not shown), the catheter (40) may include one or more lumens. For example, the catheter (40) may include a lumen through which purge fluid may travel to the motor section (30). In some embodiments, the catheter (40) may include a pre-formed bend configured to rest against a pre-determined portion of the patient's anatomy when the blood pump (10) is in a desired location.

A distal extension (100) may be operably coupled to the distal end (29) of the pump section (20). In some embodiments, this may be a direct coupling, as shown in FIG. 1A. In some embodiments, this may be an indirect coupling; that is, there may be one or more additional elements (not shown) between the distal extension and the distal end of the pump section.

A distal extension for a blood pump may include a tubular body (110) extending from a proximal end (112) to a distal end (114). The distal end may be coupled to an intermediate point (116) on the tubular body between the proximal end and the distal end to form a closed loop (120) at a distal end (103) of an atraumatic distal extension (101). As will be appreciated, the intermediate point (116) may be placed at any suitable position between the proximal end (112) and the distal end (114).

The closed loop may be formed in any appropriate manner. For example, in some embodiments, a distal end surface (118) of the tubular body (110) may be coupled to an outer surface (119) of the tubular body at the intermediate point (116). For example, in other embodiments, the tubular body (110) may be configured as one piece forming a closed loop (120).

The closed loop (120) may define an aperture (122) extending through the closed loop. Referring to FIG. 1B, the closed loop may have a central plane (124) parallel to a central axis (198) of the tubular body (110) at the proximal end (112). A central axis (126) of the aperture (122) may extend through the closed loop in a direction perpendicular to the central plane of the closed loop.

In some embodiments, the stiffness of the distal extension may be constant. In some embodiments, the distal extension may have a proximal portion (102) that has a stiffness greater than that of a distal portion (104). In other embodiments, the distal portion (104) may have a stiffness greater than that of the proximal portion (102). In some embodiments, the proximal portion (102) may have a stiffness greater than that of the tubular body (110) forming the closed loop (120).

As will be appreciated, the distal extension may be made of any suitable material. For example, in some embodiments, the distal extension may be made of a polymer material. In other embodiments, different portions of the distal extension may be made of different materials. For example, in one embodiment, one portion of the tubular body (110) may be made of a polymer material having a first stiffness and a second portion of the tubular body (110) may be made of a polymer material having a second stiffness that is greater or less than the first stiffness. As will be appreciated, the tubular body (110) may be made of any suitable number of materials and/or portions.

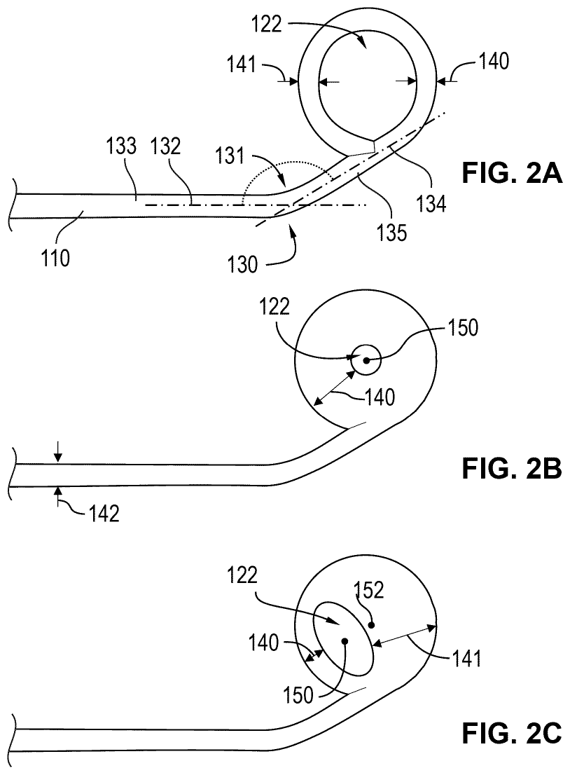

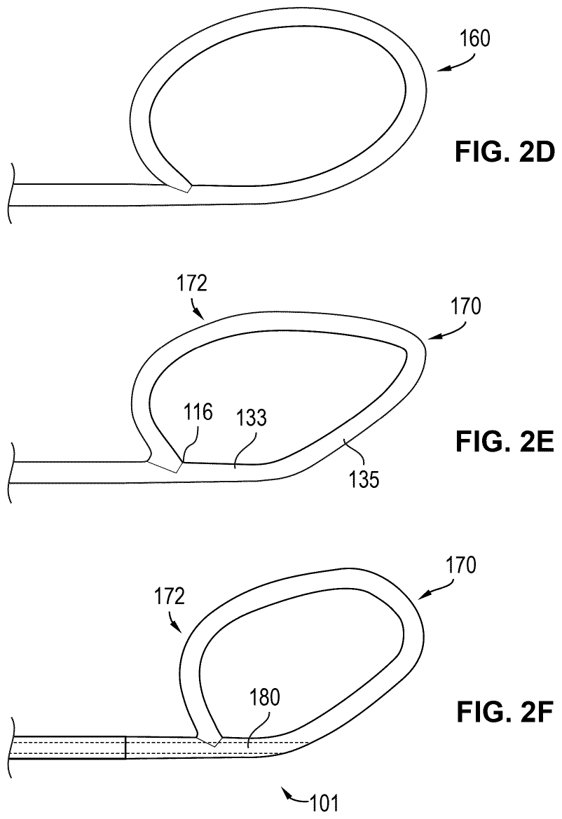

Referring to FIGS. 2A-2F, the tubular body (110) may include a bend (130). The bend may define an angle (131) between a central axis (132) of a first substantially straight section (133) of the tubular body proximal to the bend and a central axis (134) of a second substantially straight section (135) of the tubular body distal to the bend. In some embodiments, the angle is determined on the same side of tubular body that the closed loop is formed on. In some embodiments, the angle may be at least 100 degrees. In some embodiments, the angle may be at least 110 degrees.

In some embodiments, the angle (131) may be at least 120 degrees. In some embodiments, the angle may be at least 130 degrees. In some embodiments, the angle is at least 135 degrees. In some embodiments, the angle may be less than 180 degrees. In some embodiments, the angle may be less than 170 degrees. In some embodiments, the bend (130) may be configured such that a portion of the tubular body (110) will rest against at or near predetermined portion of the patient's heart when the blood pump (10) is in a desired location. For example, the bend (130) may be configured such that at least a portion of the closed loop (120) rests against a predetermined portion of the patient's heart when the blood pump (10) is in a desired location. In other examples, the bend (130) may be positioned such that a portion of the closed loop (120) is positioned at or near the apex of the left ventricle during operation of the blood pump (10).

In some embodiments, a diameter or thickness (140) of the tubular body at a first point of the closed loop may be equal to a diameter or thickness (141) of the tubular body at a second point of the closed loop (see, e.g., FIG. 2A). In some embodiments, a diameter or thickness (142) of the tubular body at the proximal end may be different from a diameter or thickness (140) of the tubular body in the closed loop (see, e.g., FIG. 2B). In some embodiments, a diameter or thickness (140) of the tubular body at a first point of the closed loop may be different from a diameter or thickness (141) of the tubular body at a second point of the closed loop (see, e.g., FIG. 2C). In some embodiments, the tubular body may have a constant thickness or diameter along the entire closed loop.

In some embodiments, the diameter or thickness (140) of the tubular body at a first point of the closed loop may increase or decrease along the length of the tubular body (110) to a diameter or thickness (141) of the tubular body at a second point of the closed loop. For example, the diameter or thickness (140) may increase or decrease continuously to the diameter or thickness (141) at a second point of the closed loop. In other embodiments, the diameter or thickness (140) of the tubular body in the closed loop may increase or decrease along the length of the tubular body to a diameter or thickness (142) of the tubular body at the proximal end. As will be appreciated, although certain geometries are described herein, the diameter or thickness may increase or decrease using any suitable geometry. For example, the diameter or thickness may increase or decrease in step-wise changes. In some embodiments, the diameter or thickness may increase or decrease in one or more segments. For example, the diameter or thickness may increase or decrease along one or both of the straight sections (133), (135). Alternatively, in other embodiments, the diameter or thickness may increase or decrease along at least a portion of the length of the tubular body forming the closed loop.

As will further be appreciated, the diameter or thickness may increase or decrease via any suitable combination of any suitable geometries. While diameter or thickness is used, there is no requirement that the tubular body have a circular cross-sectional measurement. For example, in some embodiments, the tubular body may be an oval shape and thus the diameter may refer to the largest cross-sectional measurement. As will be appreciated, although the tubular body is shown having a circular cross-sectional geometry, the tubular body may be of any suitable cross-sectional geometry.

The aperture may be configured to have any appropriate shape or design. In some embodiments, the aperture may be circular (see, e.g., FIGS. 2A-2B). In some embodiments, the aperture may be non-circular (see, e.g., FIGS. 2C-2F). In some embodiments, a central axis (150) of the aperture is centered within the closed loop (see, e.g., FIG. 2B). In some embodiments, a central axis (150) of the aperture is off-center within the closed loop (see, e.g., FIG. 2C, where a central axis (150) of the aperture does not pass through a center point (152) of the closed loop).

In some embodiments, the closed loop may be circular (see, e.g., FIGS. 2A-2C). In some embodiments, the closed loop may be non-circular (see, e.g., FIGS. 2A-2C).

In some embodiments, the closed loop may be defined by a continuous curve (160) (see, e.g., FIGS. 2A and 2D). In some embodiments, the closed loop may have one or more substantially straight sections (133), (135) (see, e.g., FIG. 2E). In some embodiments, where the distal extension includes a bend (130), the intermediate point (116) may be located proximal to the bend (see, e.g., FIGS. 2E-2F). In some embodiments, where the distal extension includes a bend, the intermediate point (116) may be located distal to the bend (see, e.g., FIG. 2A).

In some embodiments, a distal extension with a “pointier” or “sharper” distal end portion may be beneficial. Referring to FIG. 2E, in some embodiments, the closed loop may include a first point (170), furthest from the intermediate point (116), that has a radius of curvature that is less than a radius of curvature at a second point (172) closer to the intermediate point than the first point.

In some embodiments, a distal extension with a “flatter” or “less sharp” distal end portion may be beneficial. Referring to FIG. 2F, in some embodiments, the closed loop may include a first point (170), furthest from the proximal end, that has a radius of curvature that is less than a radius of curvature at a second point (172) closer to the proximal end than the first point.

The distal extension may include a lumen (180) extending through at least a portion of the atraumatic distal extension. The lumen may be configured to receive a guidewire. In some embodiments, the distal extension may be free of any lumen. In some embodiments, the distal extension may be free of a lumen configured to receive a guidewire.

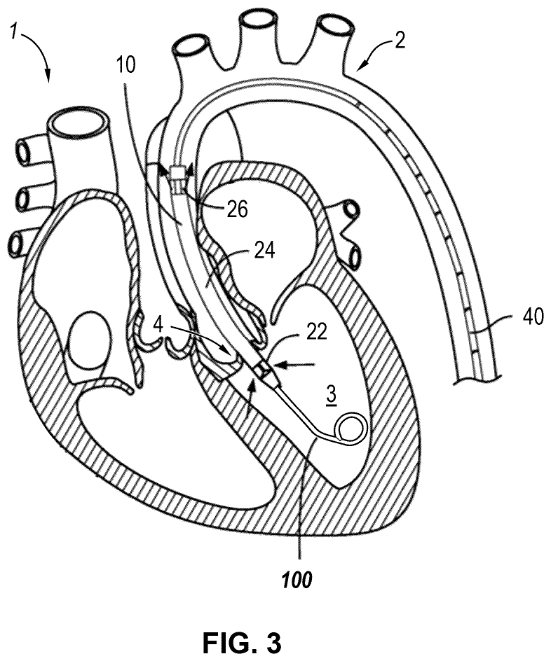

In various aspects, a method for inserting a blood pump may be provided. Referring to FIG. 3, the method may include introducing a system, which may include a blood pump (10) as disclosed herein, to a blood vessel, such as aorta (2). The blood pump may have an embodiment of a distal extension (100) as disclosed herein at a distal end of the blood pump.

The method may include advancing the system through the blood vessel, without use of a guidewire, and allowing the distal extension (100) to interact with at least one valve (4), until the blood pump (10) is disposed in a desired location (such as a left ventricle (3) of a heart (1)).

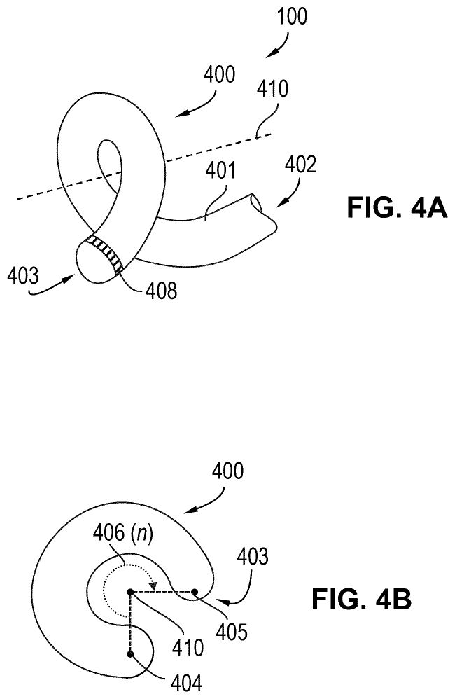

In some aspects, a helical distal extension may be provided. Referring to FIG. 4A, the distal extension (100) for a blood pump may include a tubular body (401) extending from a proximal end (402) to a distal end (403), where the tubular body defines a helical structure (400) around an axis (410). The tubular body may include a radiopaque portion (408), which may be, e.g., at or near the distal end (403). The end of the helix may be useful for, e.g., interacting with leaflets on valves (such as an aortic valve), or interacting with other medical devices. The helix shape may allow the piggybacking of the blood pump on a guide catheter, without the use of a guidewire.

Referring to FIG. 4B, from a proximal end point (404) to a distal end point (405) of the tubular body, the helical structure (400) may be configured to completes n turns (406) around the axis (410), where n is at least 0.25. In FIG. 4B, n is roughly 0.75, but as will be understood, there is no real limit to the number of turns. In some embodiments, n may be ≥0.25, ≥0.5, ≥0.75, ≥1, or ≥2. In some embodiments, n may be ≤20, ≤10, ≤9, ≤8, ≤7, ≤6, ≤5, ≤4, ≤3, ≤2, or ≤1.

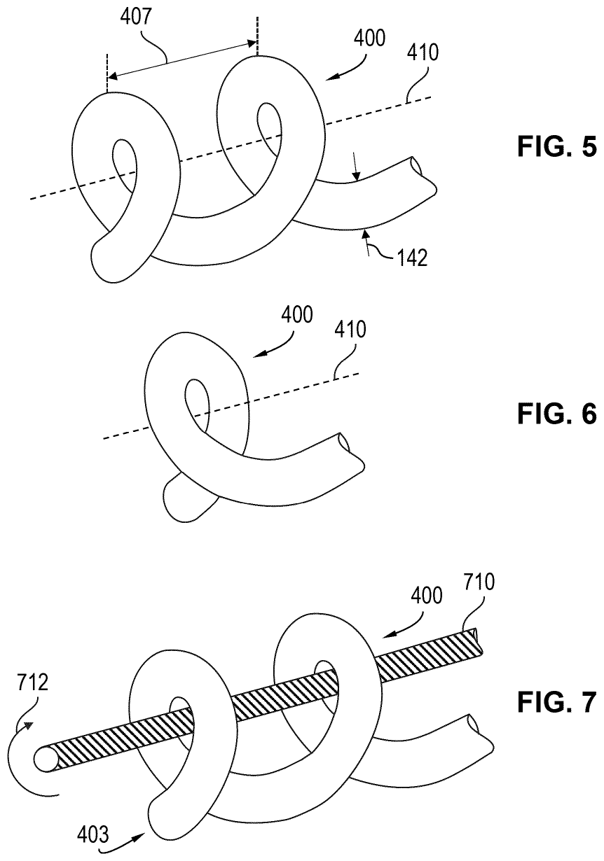

Referring to FIG. 5, the pitch (407) of the helix may be any appropriate distance. For various distal extensions, the pitch p may be greater than a diameter or thickness (142) of the tubular body. The pitch may be less than three times a diameter or thickness (142) of the tubular body. The pitch may be at least three times a diameter or thickness (142) of the tubular body. The pitch may be no more than 10 times a diameter or thickness (142) of the tubular body. In certain aspects, the pitch may be ≥1.25, ≥1.5, ≥1.75, ≥2, ≥2.5, ≥3, or ≥5 times a diameter or thickness (142) of the tubular body. In certain aspects, the pitch may be ≤20, ≤10, ≤9, ≤8, ≤7, ≤6, or ≤5 times a diameter or thickness (142) of the tubular body.

The tubular body may curl around the axis to form the helical structure either clockwise or counterclockwise, from the proximal end to the distal end. In FIGS. 4A and 5, the tubular body curls clockwise. Referring to FIG. 6, it can be seen that the tubular body may also curl counterclockwise.

In some embodiments, protrusions may extend from a surface of the tubular body to form the helical structure.

In various aspects, a method of inserting a blood pump may be provided. The method may include introducing a guide catheter to a blood vessel. Such catheters are well known in the art. The path of the guide catheter may be over the aortic arch and into the left ventricle.

The method may include introducing a blood pump to the blood vessel, the blood pump having a distal extension (100) with a helical structure (400) as disclosed herein disposed at a distal end.

The method may include advancing the blood pump through the blood vessel, without use of a guidewire. The method may include rotating the blood pump to cause the helical structure to engage the guide catheter. An example of this may be seen in FIG. 7, showing a guide catheter (710) passing through the helical structure (400). In practice, by rotating the blood pump in an appropriate direction (712), the distal end (403) of the helical structure (400) can engage the guide catheter and cause the helical structure to wrap at least partially around the guide catheter as the blood pump continues to be rotated.

The method may include rotating the blood pump to cause the helical structures to interact with one or more leaflets of the aortic valve. In the same way the distal end of the helical structure was used to engage the guide catheter, the distal end and the helical structure can also interact with leaflets of a valve, which can aid in making insertion easier, and provides some control over the positioning of the leaflets.

The method may include removing the guide catheter. For example, once a blood pump is appropriate placed within a blood vessel, the guide catheter may be removed.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

1. A distal extension for a blood pump, comprising:
a tubular body extending from a proximal end to a distal end, the distal end coupled to an intermediate point on the tubular body between the proximal end and the distal end to form a closed loop at a distal end of an atraumatic distal extension, the closed loop defining an aperture extending through the closed loop, the closed loop having a central plane parallel to a central axis of the tubular body at the proximal end;
wherein the tubular body includes a bend, the bend defining an angle between a central axis of a first substantially straight section of the tubular body proximal to the bend and a central axis of a second substantially straight section of the tubular body distal to the bend.

2. The distal extension of claim 1, wherein a central axis of the aperture extends through the closed loop in a direction perpendicular to the central plane of the closed loop.

3. The distal extension of claim 2, wherein the aperture is circular.

4. The distal extension of claim 2, wherein the aperture is non-circular.

5. The distal extension of claim 2, wherein a central axis of the aperture is off-center within the closed loop.

6. The distal extension of claim 2, wherein a central axis of the aperture is centered within the closed loop.

7. The distal extension of claim 2, wherein a diameter or thickness of the tubular body at the proximal end is different from a diameter or thickness of the tubular body in the closed loop.

8. The distal extension of claim 2, wherein a diameter or thickness of the tubular body at a first point of the closed loop is equal to a diameter or thickness of the tubular body at a second point of the closed loop.

9. The distal extension of claim 2, wherein a diameter or thickness of the tubular body at a first point of the closed loop is different from a diameter or thickness of the tubular body at a second point of the closed loop.

10. The distal extension of 2, wherein the tubular body has a constant thickness or diameter along all of the closed loop.

11. The distal extension of claim 1, wherein a distal end surface of the tubular body is coupled to an outer surface of the tubular body at the intermediate point.

12. The distal extension of claim 1, wherein the intermediate point is located proximal to the bend.

13. The distal extension of claim 1, wherein the intermediate point is located distal to the bend.

14. The distal extension of claim 1, wherein the distal extension has a proximal portion that has a stiffness greater than that of a distal portion.

15. The distal extension of claim 1, wherein the angle defined by the bend is at least 135 degrees and less than 180 degrees.

16-17. (canceled)

18. The distal extension of claim 1, wherein the closed loop includes a first point, furthest from the intermediate point, that has a radius of curvature that is less than a radius of curvature at a second point closer to the intermediate point than the first point.

19. The distal extension of claim 1, wherein a first point, furthest from the proximal end, has a radius of curvature that is less than a radius of curvature at a second point closer to the proximal end than the first point.

20-28. (canceled)

29. A distal extension for a blood pump, comprising:
a tubular body extending from a proximal end to a distal end; the distal end coupled to an intermediate point on the tubular body between the proximal end and the distal end to form a non-circular closed loop at a distal end of an atraumatic distal extension, the non-circular closed loop defining an aperture extending through the non-circular closed loop, the non-circular closed loop having a central plane parallel to a central axis of the tubular body at the proximal end.

30-53. (canceled)

54. A distal extension for a blood pump, comprising:
a tubular body extending from a proximal end to a distal end, the tubular body defining a helical structure around an axis, where, from a proximal end point to a distal end point of the tubular body, the helical structure completes n turns, where n is at least 0.25.

55-69. (canceled)

70. A method of inserting a blood pump, comprising:
introducing a guide catheter to a blood vessel;
introducing a blood pump to the blood vessel, the blood pump having a distal extension of claims 54 disposed at a distal end; and
advancing the blood pump through the blood vessel, without use of a guidewire; and
rotating the blood pump to cause the helical structure to engage the guide catheter.

71-72. (canceled)
